(19) United States
(12) Patent Application Publication
(10) Pub. No.: US 2026/0350276 A1
(43) Pub. Date: Nov. 5, 2026

(54) SIMULATION OF OCULAR FLUID FLOW BASED ON DIGITAL TWIN OF EYE

(71) Applicant: Alcon Inc., Fribourg (CH)

(72) Inventors: Sonalee Tambat, Irvine, CA (US); Paul J. Missel, Arlington, TX (US); Abolfazl Mani Irannejad, Santa Ana, CA (US)

(21) Appl. No.: 19/653,610

(22) Filed: Apr. 21, 2026

Related U.S. Application Data
(60) Provisional application No. 63/797,691, filed on Apr. 30, 2025.

(51) Int. Cl. G06F 30/28; G06F 30/27

(52) U.S. Cl. CPC G06F 30/28; G06F 30/27

(57) ABSTRACT

A system for simulating ocular fluid flow in an eye of a live patient includes a controller having a processor and tangible, non-transitory memory on which instructions are recorded. The controller is adapted to obtain respective digital images of cadaver eye sections. The controller is adapted to extract respective contours of anatomical structures in the respective digital images. A model of one or more pathways of the ocular fluid flow is generated based in part on the respective digital images. The controller is adapted to obtain patient data of the eye, including imaging data. A digital twin of the eye is generated by morphing the patient data with the model, including matching identifiable structures in the imaging data and the model. The controller is adapted to generate simulated ocular fluid flow for the eye by modeling the ocular fluid flow in the digital twin.

### INTRODUCTION

The disclosure relates generally to a system for simulating ocular fluid flow in an eye based on a digital twin of the eye, and a corresponding method. The human eye contains aqueous humor, which is a clear fluid that fills the anterior and posterior chambers of the eye, and assists in maintaining intraocular pressure. The aqueous humor is directed from the anterior chamber of the eyeball to the trabecular meshwork and Schlemm’s canal, which delivers it to the veins of the eyeball. An obstruction in the flow of aqueous humor may result in issues for the eye. Several diagnostic techniques may be employed to map the flow of aqueous humor, such as fluorescent dye injection to visualize fluid dynamics, and phase-contrast magnetic resonance imaging. However, these techniques may be burdensome and time-consuming to implement.

### SUMMARY

Disclosed herein is a system for simulating ocular fluid flow in an eye of a live patient. The system includes a controller having a processor and tangible, non-transitory memory on which instructions are recorded. Execution of the instructions by the processor causes the controller to obtain respective digital images of one or more cadaver eye sections. The controller is adapted to extract respective contours of anatomical structures in the respective digital images and generate a model of one or more pathways of the ocular fluid flow based in part on the respective digital images. Patient data of the eye is obtained, including imaging data. The controller is adapted to generate a digital twin of the eye by morphing the patient data with the model, including matching identifiable structures in the imaging data and the model. Simulated ocular fluid flow for the eye is generated by modeling the ocular fluid flow in the digital twin.

The controller may be adapted to generate optimal parameters for an ocular drainage implant based on the simulated ocular fluid flow. The optimal parameters may include a radial reference position of the ocular drainage implant in the eye. In some embodiments, the ocular drainage implant includes a base structure between a first end and a second end, the base structure including a plurality of openings for enabling the ocular fluid flow. The optimal parameters may include respective sizes of the plurality of openings and respective spines between the plurality of openings.

In some embodiments, the respective digital images are taken of a combination of the cadaver eye sections and one or more live eye stained in-vivo with imaging sensitive dye. The cadaver eye sections may be selected from a cadaver identified to have open angle glaucoma. The controller may be adapted to generate optimal parameters for patient-specific glaucoma therapy and/or a device placement plan for an ocular drainage implant based on the simulated ocular fluid flow. The anatomical structures in the respective digital images may include Schlemm’s canal, a plurality of collector channels, and trabecular meshwork. The respective digital images have a predefined minimum resolution. The predefined minimum resolution may be between about 10 nanometers to 100 nanometers. The controller may be adapted to execute a machine learning module to morph the patient data with the model. The model may be three-dimensional.

In some embodiments, the patient data includes at least one measurement of intraocular pressure of the eye. The patient data may include intraoperative optical coherence tomography data. The identifiable structures in the patient data may include a plurality of collector channels. The patient data may include imaging data. The controller is adapted to scale and align the respective digital images with the imaging data prior to morphing the patient data with the model.

Disclosed herein is a method of simulating ocular fluid flow in an eye with a system having a controller with at least one processor and at least one non-transitory, tangible memory. The method includes obtaining respective digital images of one or more cadaver eye sections and extracting respective contours of anatomical structures in the respective digital images. The respective digital images have a predefined minimum resolution. The method includes generating a model of one or more pathways of the ocular fluid flow based in part on the respective digital images, and obtaining patient data of the eye, including imaging data. A digital twin of the eye is created by morphing the patient data with the model, including matching identifiable structures in the imaging data and the model. The method includes generating simulated ocular fluid flow for the eye by modeling the ocular fluid flow in the digital twin.

The above features and advantages and other features and advantages of the present disclosure are readily apparent from the following detailed description of the best modes for carrying out the disclosure when taken in connection with the accompanying drawings.

### BRIEF DESCRIPTION OF THE DRAWINGS

FIG. 1 is a schematic illustration of a system for simulating ocular fluid flow in an eye, the system having a controller;

FIG. 2 is a schematic sectional example diagram of a portion of the eye of FIG. 1;

FIG. 3 is a schematic flowchart for a method executable by the controller of FIG. 1;

FIG. 4 is a schematic diagram illustrating an example ocular drainage implant;

FIG. 5 is a schematic fragmentary diagram illustrating the ocular drainage implant of FIG. 4 after being implanted inside an eye; and

FIG. 6 is a schematic diagram illustrating simulated ocular fluid flow for an eye with an ocular drainage implant, the fluid flow being simulated by the controller of FIG. 1.

Representative embodiments of this disclosure are shown by way of non-limiting example in the drawings and are described in additional detail below. It should be understood, however, that the novel aspects of this disclosure are not limited to the particular forms illustrated in the above-enumerated drawings. Rather, the disclosure is to cover modifications, equivalents, combinations, sub-combinations, permutations, groupings, and alternatives falling within the scope of this disclosure as encompassed, for instance, by the appended claims.

### DETAILED DESCRIPTION

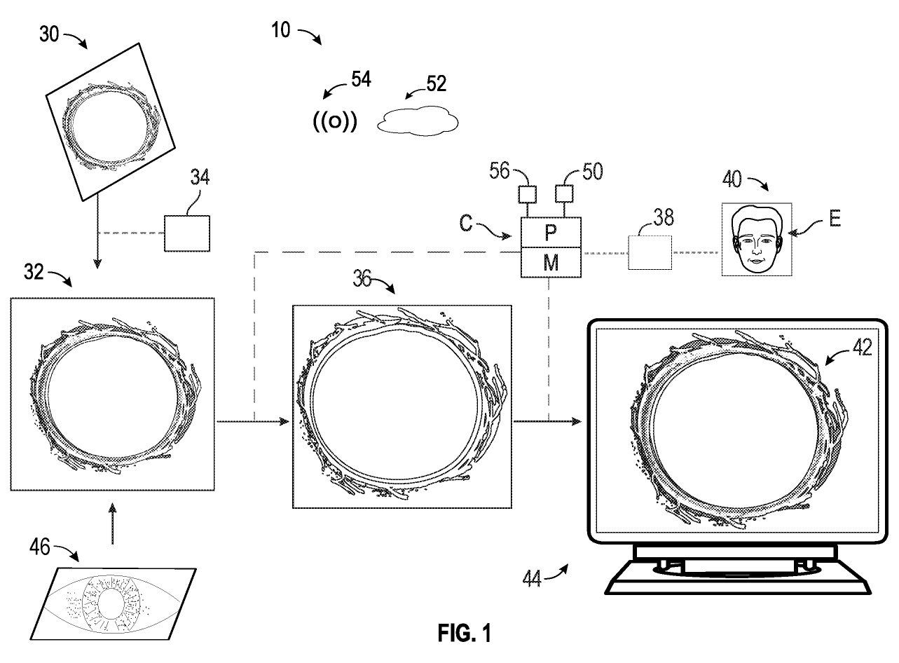

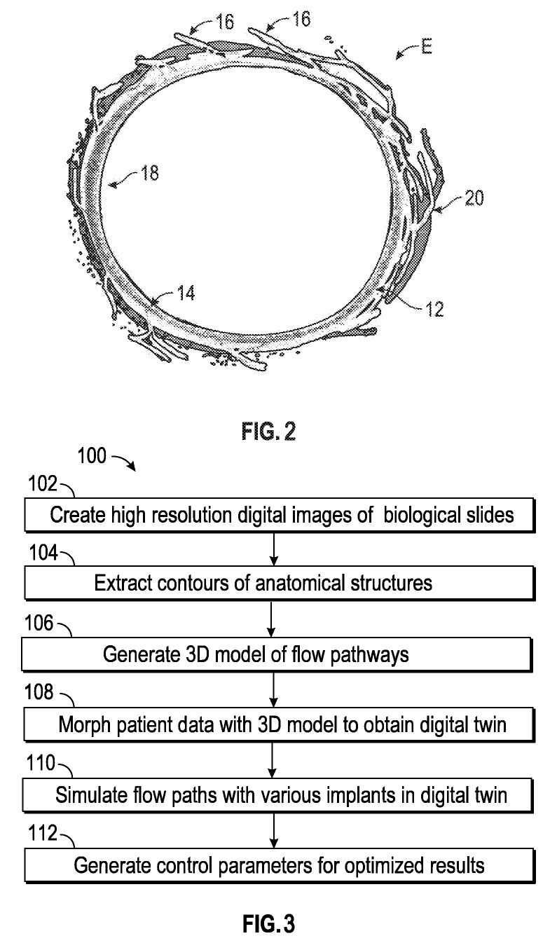

Referring to the drawings, wherein like reference numbers refer to like components, FIG. 1 schematically illustrates a system 10 for simulating ocular fluid flow in an eye E of a live patient 40. A sectional view of a portion of the eye E is shown in FIG. 2. Referring to FIG. 2, ocular fluid (i.e., aqueous humor) is directed from the anterior chamber of the eyeball to Schlemm’s canal 12 and the trabecular meshwork layer 14. Schlemm’s canal 12 is a ring-shaped channel that encircles the eye E behind the trabecular meshwork layer 14. Feature 18 denotes the boundary of the trabecular meshwork with the aqueous humor. Referring to FIG. 2, the aqueous humor flows from Schlemm’s canal 12 into a plurality of collector channels 16. Each of the collector channels 16 acts as a bridge, transferring the ocular fluid from Schlemm’s canal 12 to the episcleral veins 20. There is limited visibility in terms of ocular fluid flow between the anterior and episcleral venous regions, making it difficult to detect obstructions in the fluid flow and/or to implement remedies to offset any obstructions.

As described below, the system 10 provides a model of the eye E that extracts the ocular fluid path and tissue boundaries and enables computational modeling to demonstrate the effect of fluid obstructions on intraocular pressure. Referring to FIG. 1, the system 10 includes a controller C having at least one processor P and at least one memory M (or non-transitory, tangible computer readable storage medium) on which are recorded instructions for executing a method 100 for simulating ocular fluid flow, described below in detail with reference to FIG. 3.

As will be described below and referring to FIG. 1, the system 10 includes obtaining cadaver eye sections 30. The controller C is adapted to obtain respective digital images 32 of the cadaver eye sections 30. The digital images 32 may be taken with an imaging device 34, such as a high-resolution digital camera. Referring to FIG. 1, the controller C is adapted to generate a model 36 of one or more pathway of the ocular fluid flow based in part on the digital images 32. The model 36 may be employed to simulate the interaction of ocular drainage implants with eye structures and their effect on the resulting intraocular pressure. Referring to FIG. 1, a digital twin 42 of the eye E is created by morphing patient data 38 of the eye E of the patient 40 with the model 36. The controller C is adapted to generate simulated ocular fluid flow for the eye E of the patient 40 by simulating or modelling the ocular fluid flow in the digital twin 42, which may be viewed on a display 44.

Referring now to FIG. 3, a flow chart of method 100 executable by the controller C of FIG. 1 is shown. Method 100 need not be applied in the specific order recited herein and some blocks may be omitted. The memory M can store controller-executable instruction sets, and the processor P can execute the controller-executable instruction sets stored in the memory M.

Per block 102 of FIG. 3, the method 100 includes obtaining cadaver eye sections 30, which may be placed on slides. The cadaver eyes may be obtained through organ/tissue donation programs. The cadaver eye may be fixed with a preservative, such as formalin or glutaraldehyde to preserve the tissue structure. The preserved eyes may then be embedded in a supporting material for stability. Here, materials such as paraffin wax and plastic resins may be employed. The cadaver eye is sectioned using various methods. For example, the dissection may be performed via a microtome to create thin sections or through vibrating blades for thicker sections. Alternatively, the tissue may be frozen and dissected with a cryostat. A meticulous dissection is performed in order to preserve the delicate trabecular meshwork layer 14 that surrounds Schlemm’s canal 12. Additionally, microsurgical techniques may be used to remove some of the surrounding tissues. The eye sections may be placed on a slide and stained with various dyes to highlight the contours of Schlemm’s canal 12 and other structures. In some embodiments, the cadaver eye sections 30 are selected from a cadaver identified to have open angle glaucoma.

Also, per block 102, the controller C is adapted to obtain respective digital images 32 of the cadaver eye sections 30. The digital images 32 may be taken with an imaging device 34, such as a high-resolution digital camera. The digital images 32 are selected to have a predefined minimum resolution. In one example, the predefined minimum resolution is between about 10 nanometers to 100 nanometers. In some embodiments, the digital images 32 include a combination of images of the cadaver eye sections 30 and one or more live eye stained in-vivo with imaging sensitive dye, shown at element 46 in FIG. 1. In-vivo eye staining works by applying specialized dyes to the ocular surface. The dyes selectively bind to specific tissues or cells based on their chemical properties. For example, fluorescein highlights epithelial defects while indocyanine green has an affinity for vascular tissues. When illuminated with the appropriate wavelength of light, these dyes fluoresce or change color, making otherwise invisible structures or abnormalities clearly visible under specialized imaging equipment. The distribution of staining may be captured by a high-resolution digital camera. The live eye images may be from the patient 40, from a database of recorded live images, or a combination of both.

Advancing to block 104 of FIG. 3, the method 100 includes extracting respective contours of anatomical structures in the digital images 32. The anatomical structures in the digital images 32 may include Schlemm’s canal 12, the trabecular meshwork layer 14 (and juxtacanalicular layer), the boundary between the trabecular meshwork and the aqueous humor (denoted by feature 18 in FIG. 2), the collector channels 16 and their branching pattern and the episcleral veins 20 (see FIG. 2).

Proceeding to block 106 of FIG. 3, the controller C is adapted to generate a model 36 of one or more pathway of the ocular fluid flow based in part on the digital images 32 and the respective contours of anatomical structures obtained in block 104. Prior to morphing the patient data 38 with the model 36, the controller C is adapted to scale and align the digital images 32 and the model 36 with the patient data 38. The digital images 32 are aligned to ensure anatomical structures match across adjacent slices.

Image preprocessing filters may be applied to the digital images 32 to enhance contrast, reduce noise, and normalize brightness across the digital images 32. Generating the model 36 includes segmenting the digital images 32 based on the anatomical structures either manually, with algorithms/tools, or using deep learning approaches. In some embodiments, the model 36 is a computer-aided design (CAD) geometry model which is three-dimensional. For three-dimensional reconstruction of the model 36, the segmented structures may be interpolated between slices to create volumetric models. The resulting three-dimensional structure may be smoothed out with filtering algorithms.

Advancing to block 108 of FIG. 3, the method 100 includes morphing patient data 38 of the eye E of the patient 40 with the model 36 to create a digital twin 42 of the eye E. The patient data 38 may include at least one measurement of intraocular pressure. The patient data 38 may include imaging data, such as optical coherence tomography data, magnetic resonance imaging data, and other imaging techniques available to those skilled in the art. The controller C is adapted to match identifiable structures in the imaging data of the patient data 38 and the model 36. The identifiable structures in the patient data 38 may include Schlemm’s canal 12, the trabecular meshwork layer 14, the boundary between the trabecular meshwork and the aqueous humor (denoted by feature 18 in FIG. 2), and the collector channels 16, shown in FIG. 2.

In some embodiments, the controller C is adapted to execute a machine learning module 50 to morph the patient data 38 with the model 36. The machine learning module 50 may include a neural network trained using training datasets. The training process occurs in a closed loop or iterative fashion, with the neural network being trained until a certain criteria is met, i.e., until the discrepancy between the network outcome and ground truth reaches a point below a certain threshold. As a predefined loss function related to the training dataset is minimized, the neural network reaches convergence. The convergence signals the completion of the training. The system 10 may be configured to be “adaptive” and updated periodically after the collection of additional training data for the machine learning module 50. It is to be understood that the system 10 is not limited to a specific neural network methodology.

Referring to FIG. 1, the controller C may retrieve training datasets, patient data 38 and other data pertaining to the patient 40 from a cloud database 52 storing input data pertaining to the patient 40. The cloud database 52 may include one or more servers hosted on the Internet to store, manage, and process data. The various components of the system 10 of FIG. 1 may communicate via a wireless network 54.

Proceeding to block 110 of FIG. 3, the controller C is adapted to simulate ocular fluid flow for the eye E by simulating the ocular fluid flow in the digital twin 42. To map the fluid flow, computational fluid dynamics simulation software may be employed. Mapping the ocular fluid flow may include defining various boundary conditions such as inlet pressure, outlet pressures, fluid properties (viscosity of aqueous humor), and wall conditions. Mapping the ocular fluid flow may include solving the Navier-Stokes and Darcy’s Law equations to model the fluid flow through the ocular structures comprised of aqueous fluid or porous media respectively. In some embodiments, the controller C is adapted to execute a computational model 56 to simulate the ocular fluid flow. The controller C may employ computational fluid dynamics (CFD) using the finite element, the finite volume, or similar method, or multi-physics software to execute the computational model 56. As understood by those skilled in the art, multi-physics simulation software refers to software that can simulate models from different physical domains (e.g., interacting physical models), such as ANSYS Fluent, ANSYS CFX, COMSOL multi-physics or other similar software. Additionally, after simulation of the ocular fluid flow, visualization tools may be employed to generate streamlines, velocity vectors, and pressure maps to analyze the fluid dynamics.

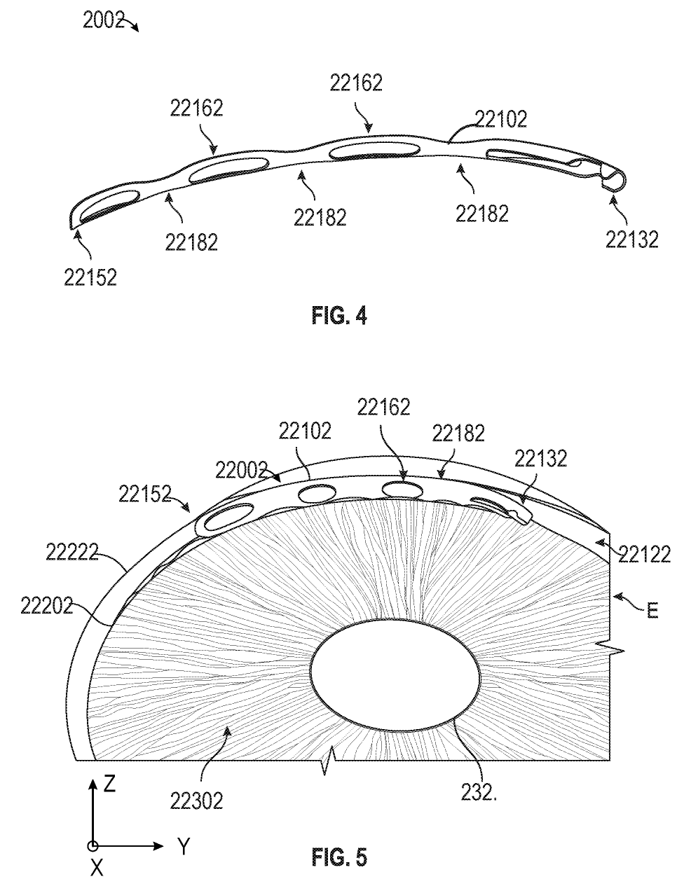

Referring to FIG. 1, the digital twin 42 may be employed to design an implanted device that moves or facilitates the movement of ocular fluid in a preferred direction based on idiosyncratic patient anatomy. In other words, the system 10 allows for virtually simulating the interaction of an ocular drainage implant with the eye E. An example ocular drainage implant 200 is shown in FIGS. 4-5. The ocular drainage implant 200 is used to help manage the flow of aqueous humor and maintain a consistent intraocular pressure. Referring to FIGS. 4-5, the ocular drainage implant 200 includes a base structure 210 between a first end 213 and a second end 215. Referring to FIGS. 4-5, the base structure 210 includes a plurality of openings 216 that allow ocular fluid to flow through or across. The openings 216 are separated by respective spines 218. The respective sizes of the openings 216 may be non-uniform or varied across the base structure 210. The ocular drainage implant 200 may be made of nickel and a titanium alloy. It is understood that other structures or types of ocular implants may be employed.

FIG. 5 illustrates the ocular drainage implant 200 after being positioned inside the eye E. Referring to FIG. 5, the ocular drainage implant 200 may be inserted in Schlemm’s canal 212, between an inner layer 220 and an outer layer 222 of the Schlemm’s canal of eye E. The ocular drainage implant 200 may be fashioned as a flexible metal tube that mechanically expands the width of the Schlemm’s canal 212. The ocular drainage implant 200 creates a lower-resistance path for the ocular fluid, enabling direct entry into Schlemm’s canal 212 and clearance through the collector channels 16. This helps lower intraocular pressure, which assists in managing progression of glaucoma in the patient 40. Also shown in FIG. 5 are the iris 230 and pupil 232.

The simulated fluid flow may be employed as a preoperative diagnostic tool for designing the ocular drainage implant 200. For example, the fluid flow simulation may be performed with different types of ocular drainage implants to compare the improvement in drainage. Advancing to block 112 of FIG. 3, the method 100 includes generating optimal parameters for the ocular drainage implant 200 based on the simulated ocular fluid flow. The optimal parameters may include a radial reference position (relative to the ZY plane) of the ocular drainage implant 200 in the eye E. The optimal parameters may include the respective sizes of the plurality of openings 216 and respective sizes of the spines 218. The optimal parameters may include the angle subtended between the first end 213 and second end 215 of the ocular drainage implant 200. In some embodiments, the ocular drainage implant 200 may be produced with a 3D printer based on patient anatomy.

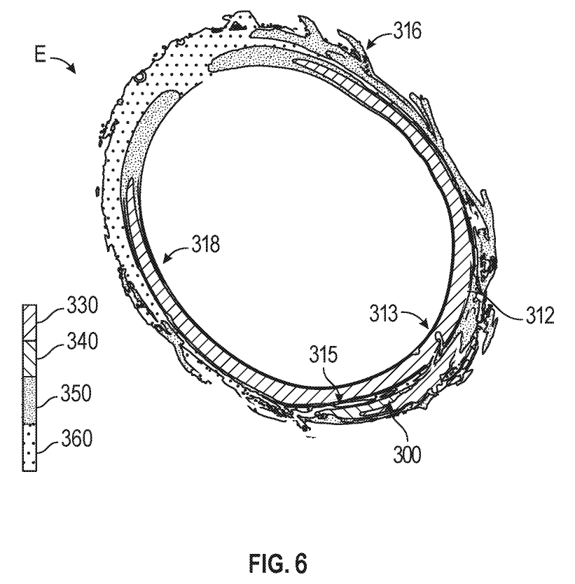

The method 100 may include conducting a first fluid flow simulation without any implants, followed by a second fluid flow simulation with an ocular drainage implant, to illustrate the improved drainage and pressure-lowering effect. Referring now to FIG. 6, an example simulated ocular fluid flow for an eye E (that is implanted with an ocular drainage implant 300) is shown. Referring to FIG. 6, the ocular drainage implant 300 is placed in Schlemm’s canal 312, and extends between a first end 313 and a second end 315. Also shown in FIG. 6 are collector channels 316, and a shell of aqueous humor of the eye E, which lies immediately outside and is adjoining to the trabecular meshwork layer 14. Feature 318 denotes the inner surface of this shell of aqueous humor.

In the simulation shown in FIG. 6, the computational domain included the trabecular meshwork layer 14, Schlemm’s Canal 12, the collector channels 16, and the thin band or shell of aqueous humor inside and adjoining the trabecular meshwork layer 14. The inner boundary of the aqueous shell, feature 318, provided a boundary on which a flow inlet could be placed to allow the generation of aqueous humor as required for a computational fluid dynamic simulation. It is understood that other parameters or features may be employed. Referring to the legend on the left side of FIG. 6, the region 330 of highest fluid velocity is shown with a hatched (forward-slash) pattern. The region 340 of second-highest fluid velocity is shown with a hatched (back-slash) pattern. The region 350 of third-highest fluid velocity is shown with a densely dotted pattern. The region 360 of fourth-highest fluid velocity is shown with a sparsely dotted pattern.

In summary, the system 10 enables the development of a three-dimensional, patient-specific digital twin 42 of an eye E for simulating the path of ocular fluid. The controller C reconstructs the fluid path of Schlemm's canal 12 and collector channels 16, incorporating additional tissues. The system 10 further allows for virtually simulating the interaction of an ocular drainage implant 200 (or 300) with the eye E. The technical advantage here is that it enables personalized placement of the ocular drainage implant 200 in a specific radial region of the eye E, such as for example positioning the ocular drainage implant 200 relatively more nasally versus temporally or wherever needed to allow access to a greater number of collector channels, depending upon the patient’s specific ocular anatomy. The system 10 may be used to provide patient specific glaucoma therapy and device placement plan to the surgeon. By simulating various scenarios, the need for trial-and-error approaches in treatments and surgeries is reduced improving patient outcomes.

The wireless network 54 of FIG. 1 may be a bus implemented in various ways, such as for example, a serial communication bus in the form of a local area network. The local area network may include, but is not limited to, a Controller Area Network (CAN), a Controller Area Network with Flexible Data Rate (CAN-FD), Ethernet, blue tooth, WIFI and other forms of data connection. The wireless network 54 may be a Wireless Local Area Network (LAN) which links multiple devices using a wireless distribution method, a Wireless Metropolitan Area Networks (MAN) which connects several wireless LANs or a Wireless Wide Area Network (WAN). Other types of connections may be employed.

The controller C of FIG. 1 includes a computer-readable medium (also referred to as a processor-readable medium), including a non-transitory (e.g., tangible) medium that participates in providing data (e.g., instructions) that may be read by a computer (e.g., by a processor of a computer). Such a medium may take many forms, including, but not limited to, non-volatile media and volatile media. Non-volatile media may include, for example, optical or magnetic disks and other persistent memory. Volatile media may include, for example, dynamic random-access memory (DRAM), which may constitute a main memory. Such instructions may be transmitted by one or more transmission media, including coaxial cables, copper wire and fiber optics, including the wires that comprise a system bus coupled to a processor of a computer. Some forms of computer-readable media include, for example, a floppy disk, a flexible disk, hard disk, magnetic tape, other magnetic medium, a CD-ROM, DVD, other optical medium, a physical medium, a RAM, a PROM, an EPROM, a FLASH-EEPROM, other memory chip or cartridge, or other medium from which a computer can read.

Look-up tables, databases, data repositories or other data stores described herein may include various kinds of mechanisms for storing, accessing, and retrieving various kinds of data, including a hierarchical database, a set of files in a file storage system, an application database in a proprietary format, a relational database management system (RDBMS), etc. Each such data store may be included within a computing device employing a computer operating system such as one of those mentioned above and may be accessed via a network in one or more of a variety of manners. A file system may be accessible from a computer operating system and may include files stored in various formats. An RDBMS may employ the Structured Query Language (SQL) in addition to a language for creating, storing, editing, and executing stored procedures, such as the PL/SQL language mentioned above.

The flowchart shown in the FIGS. illustrates an architecture, functionality, and operation of possible implementations of systems, methods, and computer program products according to various embodiments of the present disclosure. In this regard, each block in the flowchart or block diagrams may represent a module, segment, or portion of code, which comprises one or more executable instructions for implementing the specified logical function(s). It will also be noted that each block of the block diagrams and/or flowchart illustrations, and combinations of blocks in the block diagrams and/or flowchart illustrations, may be implemented by specific purpose hardware-based systems that perform the specified functions or acts, or combinations of specific purpose hardware and computer instructions. These computer program instructions may also be stored in a computer-readable medium that can direct a controller or other programmable data processing apparatus to function in a particular manner, such that the instructions stored in the computer-readable medium produce an article of manufacture including instructions to implement the function/act specified in the flowchart and/or block diagram blocks.

The numerical values of orders (e.g., of quantities or conditions) in this specification, including the appended claims, are to be understood as being modified in each respective instance by the term “about” whether or not “about” actually appears before the numerical value. “About” indicates that the stated numerical value allows some slight imprecision (with some approach to exactness in the value; about or reasonably close to the value; nearly). If the imprecision provided by “about” is not otherwise understood in the art with this ordinary meaning, then “about” as used herein indicates at least variations that may arise from ordinary methods of measuring and using such orders. In addition, disclosure of ranges includes disclosure of each value and further divided ranges within the entire range. Each value within a range and the endpoints of a range are hereby disclosed as separate embodiments.

The detailed description and the drawings or FIGS. are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims. Furthermore, the embodiments shown in the drawings, or the characteristics of various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments fall within the framework of the scope of the appended claims.

What is claimed is:

1. A system for simulating ocular fluid flow in an eye of a live patient, the system comprising:
a controller having a processor and tangible, non-transitory memory on which instructions are recorded, execution of the instructions by the processor causing the controller to:
obtain respective digital images of one or more cadaver eye sections, the respective digital images having a predefined minimum resolution;
extract respective contours of anatomical structures in the respective digital images;
generate a model of one or more pathways of the ocular fluid flow based in part on the respective digital images;
obtain patient data of the eye, including imaging data;
generate a digital twin of the eye by morphing the patient data with the model, including matching identifiable structures in the imaging data and the model; and
generate simulated ocular fluid flow for the eye by modeling the ocular fluid flow in the digital twin.

2. The system of claim 1, wherein the controller is adapted to generate optimal parameters for an ocular drainage implant based on the simulated ocular fluid flow.

3. The system of claim 2, wherein the optimal parameters include a radial reference position of the ocular drainage implant in the eye.

4. The system of claim 2, wherein:
the ocular drainage implant includes a base structure between a first end and a second end, the base structure including a plurality of openings for enabling the ocular fluid flow; and
the optimal parameters include respective sizes of the plurality of openings and respective spines between the plurality of openings.

5. The system of claim 1, wherein the patient data includes at least one measurement of intraocular pressure of the eye.

6. The system of claim 1, wherein the patient data includes intraoperative optical coherence tomography data and the identifiable structures in the patient data include a plurality of collector channels.

7. The system of claim 1, wherein the controller is adapted to execute a machine learning module to morph the patient data with the model, the model being three-dimensional.

8. The system of claim 1, wherein:
the patient data includes imaging data; and
the controller is adapted to scale and align the respective digital images with the imaging data prior to morphing the patient data with the model.

9. The system of claim 1, wherein:
the one or more cadaver eye sections are selected from a cadaver identified to have open angle glaucoma; and
the controller is adapted to generate optimal parameters for patient-specific glaucoma therapy and/or a device placement plan for an ocular drainage implant based on the simulated ocular fluid flow.

10. The system of claim 1, wherein the anatomical structures in the respective digital images include Schlemm’s canal, a plurality of collector channels, and trabecular meshwork.

11. The system of claim 1, wherein the predefined minimum resolution is between about 10 nanometers to 100 nanometers.

12. A method of simulating ocular fluid flow in an eye of a live patient with a system having a controller with at least one processor and at least one non-transitory, tangible memory, the method comprising:
obtaining respective digital images of one or more cadaver eye sections, the respective digital images having a predefined minimum resolution;
extracting respective contours of anatomical structures in the respective digital images;
generating a model of one or more pathways of the ocular fluid flow based in part on the respective digital images;
obtaining patient data of the eye, including imaging data;
creating a digital twin of the eye by morphing the patient data with the model, including matching identifiable structures in the imaging data and the model; and
generating simulated ocular fluid flow for the eye by modeling the ocular fluid flow in the digital twin.

13. The method of claim 12, further comprising:
generating optimal parameters for an ocular drainage implant based on the simulated ocular fluid flow, via the controller; and
selecting the optimal parameters to include a radial reference position of the ocular drainage implant in the eye.

14. The method of claim 13, further comprising:
configuring the ocular drainage implant with a base structure having a first end and a second end, the base structure including a plurality of openings for the fluid flow; and
selecting the optimal parameters to include respective sizes of the plurality of openings and respective spines between the plurality of openings.

15. The method of claim 12, further comprising:
selecting the patient data to include intraoperative optical coherence tomography data.

16. The method of claim 12, further comprising:
executing a machine learning module to morph the patient data with the model, via the controller, the model being a three-dimensional model; and
selecting the patient data to include at least one measurement of intraocular pressure.

17. The method of claim 12, further comprising:
selecting the patient data to include imaging data; and
scaling and aligning the respective digital images with the imaging data prior to morphing the patient data with the model, via the controller.

18. The method of claim 12, further comprising:
selecting the one or more the cadaver eye sections from a cadaver identified to have open angle glaucoma.

19. The method of claim 12, further comprising:
setting the predefined minimum resolution to be between about 10 nanometers to 100 nanometers; and
selecting the anatomical structures in the respective digital images to include Schlemm’s canal, a plurality of collector channels, and trabecular meshwork.

20. A system for simulating ocular fluid flow in an eye of a live patient, the system comprising:
a controller having a processor and tangible, non-transitory memory on which instructions are recorded, execution of the instructions by the processor causing the controller to:
obtain respective digital images of one or more cadaver eye sections or a combination of one or more live eye stained in-vivo with imaging sensitive dye and one or more cadaver eye sections, the respective digital images having a predefined minimum resolution;
extract respective contours of anatomical structures in the respective digital images, including a location of at least one collector channel;
generate a model of one or more pathways of the ocular fluid flow based in part on the respective digital images;
obtain patient data of the eye, including imaging data;
create a digital twin of the eye by morphing the patient data with the model, including matching identifiable structures in the imaging data and the model;
generate simulated ocular fluid flow for the eye by modeling the ocular fluid flow in the digital twin;
wherein the controller is adapted to generate optimal parameters for an ocular drainage implant based on the simulated ocular fluid flow;
wherein the ocular drainage implant includes a base structure having a first end and a second end, the base structure including a plurality of openings for the ocular fluid flow; and
wherein the optimal parameters include a radial reference position of the ocular drainage implant in the eye, and respective sizes of the plurality of openings.
